(19) United States
(12) Patent Application Publication
(10) Pub. No.: US 2009/0280483 A1
(43) Pub. Date: Nov. 12, 2009

(54) Methods for Screening Interleukin-6 (IL-6) Signal Transduction Inhibitors

(76) Inventors: Toshio Kitamura, Tokyo (JP); Toshiyuki Kawashima, Tokyo (JP)

Correspondence Address: BIRCH STEWART KOLASCH & BIRCH, PO BOX 747, FALLS CHURCH, VA, 22040-0747, US

(21) Appl. No.: 12/085,324

(22) PCT Filed: Nov. 22, 2005
(86) PCT No.: PCT/JP2005/021485

§ 371 Date: May 21, 2008

(51) Int. Cl. C12Q 1/02; C12Q 1/68; C12Q 1/48; C07C 49/255; C07C 49/213; C07C 215/20; C07C 275/28; C07D 333/68; C07D 277/38

(52) U.S. Cl. 435/6; 435/29; 435/15; 568/334; 568/331; 564/363; 564/55; 549/57; 548/190

(57) ABSTRACT

The present invention provides methods for screening compounds which inhibit activation of a member of the IL-6 signaling pathways, comprising: (a) a positive screening step using a cell capable of being killed by IL-6 stimulation to select compounds which inhibit death of the cell when it is stimulated by IL-6; and then (b) a biochemical screening step to further select compounds which inhibit activation of a member of the IL-6 signaling pathways by a biochemical means from the compounds selected in step (a). The present invention also provides compounds which inhibit activation of a member of the IL-6 signaling pathways identified using said methods.

### TECHNICAL FIELD

The present invention relates to methods for screening compounds which inhibit activation of a member of the interleukin-6 (IL-6) signaling pathways, and compounds inhibiting activation of a member of the IL-6 signaling pathways identified using the screening methods.

### BACKGROUND ART

Interleukin-6 (IL-6) was originally discovered as a cytokine inducing the terminal differentiation of B cells into antibody-producing cells, and is known to be produced/secreted in various cell types such as T cells, B cells, macrophages, fibroblasts, etc. When IL-6 binds to its receptor on the cell membrane IL-6Ra, it induces association between IL-6Ra and a transmembrane protein gp130 and dimerization between gp130 molecules. Members belonging to the JAK (Janus kinase) family such as JAK1, JAK2, etc. are constitutively associated in the intracellular domain of gp130, and JAKs associated with gp130 via dimerization of gp130 come close to each other, suggesting that JAKs are activated by mutual phosphorylation of tyrosine residues. Activated JAKs further phosphorylate tyrosine residues in the intracellular domain of gp130 and various signal molecules, thereby activating these molecules.

STAT (signal transducer and activator of transcription) family members have a SH2 (src homology 2) domain recognizing phosphorylated tyrosine residues, and they bind to gp130 phosphorylated on tyrosine residues via the domain so that they are phosphorylated on tyrosine residues by JAKs. The phosphorylated STATs are released from gp130, and the SH2 domains of the released STATs then bind to phosphorylated tyrosine residues of other STATs, thereby forming STAT homodimers or heterodimers. The dimerized STATs translocate into the nucleus and bind to responsive sequences of various genes together with other gene regulatory proteins to promote transcription of the target genes. It is known that Shc and Shp also undergo phosphorylation on gp130 in a manner similar to the phosphorylation of STATs. The PI3K- and Akt-mediated pathway and Ras/MAPK pathway downstream of these molecules influence various aspects such as cell viability and differentiation (Non-patent document 1-4).

It has been known that molecules of the IL-6 signaling pathways are activated in many pathologies including multiple myeloma, Castleman's tumor, chronic rheumatoid arthritis, etc., and therefore, inhibitors for activation of a member of the IL-6 signaling pathways are expected to provide therapeutic drugs for these diseases. Especially, inhibitors of STAT3 appear to be potential broad spectrum anti-cancer agents because it is known that STAT3 is activated in many cancer cells and that its activation is involved in cell transformation. Moreover, it has been reported that mutations inducing constitutive activation of JAK2 are responsible for many cases of myeloproliferative disorder (MPD) (Non-patent document 5-8).

These facts suggest that methods for efficiently screening compounds inhibiting the Il-6 signaling pathways may lead to development of pharmaceutical drugs against various diseases including cancer.

Conventional screening of low-molecular compound libraries not only for IL-6 signal inhibitors has often relied on negative screening, e.g., by selecting compounds inhibiting cell proliferation (Non-patent document 9-11). Thus, there are demands to establish methods for efficiently screening compounds inhibiting the activation of a member of the IL-6 signaling pathways rather than relying on negative screening only.

Non-patent document 1: Nakajima K, Yamanaka Y, Nakae K, et al. A central role for Stat3 in IL-6-induced regulation of growth and differentiation in M1 leukemia cells. EMBO J. 1996; 15: 3651-3658.

Non-patent document 2: Kawashima, T., Hirose, K., Satoh, T., Kaneko, A., Ikeda, Y., Kaziro, Y., Nosaka, T., and Kitamura, T. (2000). MgcRacGAP is involved in the control of growth and differentiation of hematopoietic cells. Blood 96, 2116-2124.

Non-patent document 3: Kawashima, T., Murata, K., Akira, S., Tonozuka, Y., Minoshima, Y., Feng, S., Kumagai, H., Tsuruga, H., Ikeda, Y., Asano, S., et al. (2001). STAT5 induces macrophage differentiation of M1 leukemia cells through activation of IL-6 production mediated by NF-kappaB p 65. J. Immunol. 167, 3652-3660.

Non-patent document 4: Tonozuka Y et al. Blood 104 (12) 3550-7 (2004).

Non-patent document 5: James C, Ugo V, Le Couedic J P, Staerk J, Delhommeau F, Lacout C, Garcon L, Raslova H, Berger R, Bennaceur-Griscelli A, Villeval J L, Constantinescu S N, Casadevall N, Vainchenker W. A unique clonal JAK2 mutation leading to constitutive signalling causes polycythaemia vera. Nature. 2005; 434: 1144-1148.

Non-patent document 6: Kralovics R, Passamonti F, Buser A S, Teo S S, Tiedt R, Passweg J R, Tichelli A, Cazzola M, Skoda R C. A gain-of-function mutation of JAK2 in myeloproliferative disorders. N Engl J. Med. 2005; 352: 1779-1790.

Non-patent document 7: Levine R L, Wadleigh M, Cools J, Ebert B L, Wernig G, Huntly B J, Boggon T J, Wlodarska I, Clark J J, Moore S, Adelsperger J, Koo S, Lee J C, Gabriel S, Mercher T, D'Andrea A, Frohling S, Dohner K, Marynen P, Vandenberghe P, Mesa R A, Tefferi A, Griffin J D, Eck M J, Sellers W R, Meyerson M, Golub T R, Lee S J, Gilliland D G. Cancer Cell. 2005; 7: 387-397.

Non-patent document 8: Zhao R, Xing S, Li Z, Fu X, Li Q, Krantz S B, Zhao Z J. Identification of an acquired JAK2 mutation in polycythemia vera. J Biol. Chem. 2005; 280: 22788-22792.

Non-patent document 9: Rosania G R, Merlie J Jr, Gray N, Chang Y T, Schultz P G, Heald R. A cyclin-dependent kinase inhibitor inducing cancer cell differentiation: biochemical identification using Xenopus egg extracts. Proc Natl Acad Sci USA. 1999; 96: 4797-4802.

Non-patent document 10: Murata K, Kumagai H, Kawashima T, Tamitsu K, Irie M, Nakajima H, Suzu S, Shibuya M, Kamihira S, Nosaka T, Asano S, and Kitamura T. Selective cytotoxic mechanism of GTP-14564, a novel tyrosine kinase inhibitor in leukemia cells expressing a constitutively active Fms-like tyrosine kinase 3 (FLT3). J. Biol. Chem. 2003; 278: 32892-328980.

Non-patent document 11: Winter-Vann A M, Baron R A, Wong W, dela Cruz J, York J D, Gooden D M, Bergo M O, Young S G, Toone E J, Casey P J. A small-molecule inhibitor of isoprenylcysteine carboxyl methyltransferase with antitumor activity in cancer cells. Proc Natl Acad Sci USA. 2005; 102: 4336-41.

Non-patent document 12: Shimizu S et al. Blood 72 (5) 1826-1828 (1988).

Non-patent document 13: Burger R et al. Hematol J. 2 (1) 42-53 (2001).

### DISCLOSURE OF THE INVENTION

### Problems to be Solved by the Invention

An object of the present invention is to provide a method for screening compounds which inhibits activation of a member of the IL-6 signaling pathways.

A method of the present invention comprises:

(a) a positive screening step using a cell capable of being killed by IL-6 stimulation to select compounds which inhibit death of the cell when stimulated by IL-6; and then

(b) a biochemical screening step to further select compounds inhibiting activation of a member of the IL-6 signaling pathways by a biochemical means from the compounds selected in step (a).

In the method of the present invention, the cell capable of being killed by IL-6 stimulation used in the positive screening step is preferably derived from mice or humans, more preferably a clone derived from murine M1 cells or a genetically engineered cell.

In the method of the present invention, a target molecule of compounds inhibiting activation of a member of the IL-6 signaling pathways is preferably a member of the STAT3-mediated pathway, a member of the RAS-mediated pathway or a member of the PI3K- and Akt-mediated pathway.

In the method of the present invention, the biochemical means in the biochemical screening step is preferably any method selected from the group consisting of Western blotting, kinase assay, transcriptional activity assay, surface plasmon resonance, immunoprecipitation, cell staining, gene expression analysis using gene chips, Northern blotting, and RT-PCR or a combination thereof.

The method of the present invention further comprises, between the positive screening step and the biochemical screening step, a negative screening step using a cell capable of proliferating by IL-6 stimulation to select compounds inhibiting IL-6-dependent proliferation of the cell from the compounds selected in the positive screening step.

In the method of the present invention, the cell capable of proliferating with IL-6 stimulation used in the negative screening step is preferably derived from humans, more preferably an IL-6-dependent tumor cell line.

Another object of the present invention is to provide compounds which inhibit the activation of a member of the IL-6 signaling pathways obtained by the screening methods of the present invention.

### Means for Solving the Problems

As a result of intensive studies to solve the problems above, we identified compounds which inhibit activation of a member of the IL-6 signaling pathways by methods for screening compounds inhibiting activation of a member of the IL-6 signaling pathways and finally achieved the present invention.

### I. Methods for Screening Compounds Inhibiting the Activation of a Member of the IL-6 Signal Transduction System

The present invention provides a method for screening compounds which inhibit activation of a member of the IL-6 signaling pathways, comprising: (a) a positive screening step using a cell capable of being killed by IL-6 stimulation to select compounds which inhibit death of the cell when stimulated by IL-6; and then (b) a biochemical screening step to further select compounds which inhibit activation of a member of the IL-6 signaling pathways by a biochemical means from the compounds selected in step (a). To further improve the precision of screening, the method may further comprise, between the positive screening step (a) and the biochemical screening step (b), a negative screening step using a cell capable of proliferating with IL-6 stimulation to select compounds which inhibit IL-6-dependent proliferation of the cell from the compounds selected in step (a).

Specifically, the method for screening compounds which inhibit activation of a member of the IL-6 signaling pathways invented by the present inventors is performed by identifying compounds which inhibit IL-6-stimulated death of a cell capable of being killed by IL-6 in the positive screening step. It is very unlikely that this positive screening step will produce false positive results by the cytotoxicity of compounds, because the cell proliferates only in the presence of a compound which inhibit activation of a member of the IL-6 signaling pathways. In fact, compounds having passed the positive screening step were 0.142% or less in a pilot experiment.

As described in the examples below, 3 of 18 compounds having passed the positive screening step, i.e., Compounds 2, 13 and 16 specifically inhibited IL-6-induced activation of STAT3. Thus, it is thought that the 3 of 18 compounds inhibited the activation of STAT3 and 4 compounds inhibited the activation of another molecule in the IL-6 signaling pathways. All of them inhibited IL-6-dependent proliferation of human Lennert's T cell lymphoma cell line (KT-3 cells). As a result, the proportion of finally positive compounds among the compounds having passed the positive screening step was as high as 38.9% ( 7/18). All of these facts show effectiveness of this positive screening. Moreover, compounds which inhibit activation of a member of the IL-6 signaling pathways can be more reliably selected by using a biochemical means such as Western blotting in the biochemical screening step. This allows evaluation of which member (e.g., cytokine, receptor, kinase, transcription factor, etc.) of the IL-6 signaling pathways is inhibited from activation by the compounds selected by using the positive screening step.

Furthermore, between the positive screening step and the biochemical screening step, a negative screening step can be performed on the basis of the proliferation inhibition of a cell capable of proliferating by IL-6 stimulation to narrow down compounds which inhibit the IL-6 signaling pathways.

The “IL-6 signaling pathways” refers to a signal cascade triggered by a secretary protein IL-6, which binds to its receptor IL-6Rato activate an intracellular JAK, which in turn phosphorylates another protein. As shown in FIG. 1, the STAT-mediated pathway, Ras-mediated pathway, and PI3K- and Akt-mediated pathway, etc. are known. In embodiments of the present invention, therefore, the “member of the IL-6 signaling pathways” of the present invention comprises any one molecule or a group of molecules shown in FIG. 1. Preferably, it is a member of the STAT3-mediated pathway, a member of the RAS-mediated pathway, or a member of the PI3K- and Akt-mediated pathway.

The expression “inhibit activation of a member” includes any embodiment in which the normal function (biological function) of the member is inhibited, including, but not specifically limited to, inhibition of phosphorylation/dephosphorylation of the member, inhibition of kinase/phosphatase activity, inhibition of binding to another member, and inhibition of intracellular translocation, etc. The “inhibition” includes not only complete inhibition but also partial inhibition of activation of the member and reduction of the activation.

### i) Positive Screening Step

The positive screening step in the screening method of the present invention uses a cell capable of being killed by IL-6 stimulation to select compounds inhibiting the death of the cell when it is stimulated by IL-6.

In preferred embodiments of the positive screening step of the present invention, the cell capable of being killed by IL-6 stimulation used is derived from mice or humans.

In more preferred embodiments of the positive screening step of the present invention, the cell capable of being killed by IL-6 stimulation used is a clone derived from murine M1 cells or a genetically engineered cell.

The “cell capable of being killed by IL-6 stimulation” refers to a cell capable of being completely killed by adding IL-6 into the culture medium during cell culture. Those skilled in the art can readily understand/appreciate such a cell and can readily prepare it by selection, modification, etc. from a known cultured cell line or by genetic engineering from a known cell. Preferably, it is derived from, but not limited to, mice or humans. More preferably, it is a clone derived from murine M1 cells (M1 clone), or a genetically engineered cell. The M1 clone as used herein refers to a clone obtained by selecting clones susceptible to IL-6-induced death from known M1 murine myeloid leukemia cells (ATCC No. TIB-192, etc.). This M1 clone can be selected on the basis of death under conditions of an IL-6 concentration in the culture medium of 10 ng/ml, preferably 7.5 ng/ml, more preferably 5 ng/ml.

The “genetically engineered cell” refers to a cell prepared by a genetic engineering method to contain a DNA construct having a suicide gene downstream of a promoter sequence under the control of a transcription factor located downstream of the IL-6 signal transduction system, such as STAT3, c-Jun, ATF-2 or Elk-1. The suicide gene may also be a gene producing a cytotoxic substance such as HSV-tk, or a gene inducing apoptosis such as p53. Methods for preparing such a construct containing a gene of interest downstream of a specific promoter sequence and transiently expressing it, or methods for inserting the gene into the genome and constitutively expressing it are well known in the art.

The expression “inhibiting the death of a cell” means, without any limitation, that 10% or more, preferably 30% or more, more preferably 50% or more, still more preferably 90% or more of a cell in culture survives. The cell viability can be determined by using ordinary knowledge in the art, e.g., using FACS (described in the examples), staining with trypan blue, etc.

In the present positive screening step, compounds which inhibit activation of a member of the IL-6 signaling pathways can be screened from a compound library by adding each compound at a concentration of 20 μM, preferably 5 μM, more preferably 1.25 μM, still more preferably 0.3 μM into a medium in which a cell capable of being killed by IL-6 stimulation is to be cultured, and then adding IL-6 at a concentration of 5 ng/ml, preferably 10 ng/ml, more preferably 20 ng/ml to select compounds which allow the cell to survive.

Conventional negative screening (screening using a cell capable of proliferating with IL-6 stimulation to select compounds which inhibiting IL-6-dependent proliferation of the cell) had the problem that it was unknown whether the inhibition of cell proliferation is caused by the action of compounds or cell inhibition. However, compounds selected by the positive screening step of the present invention can be said to have no cytotoxicity and to have the function of inhibiting the activation of a member of the IL-6 signaling pathways. These properties are desirable because compounds inhibiting activation of a member of the IL-6 signaling pathways are expected for use as anti-cancer agents or the like in pharmaceutical compositions.

ii) Biochemical screening step

In the biochemical screening step of the present invention, compounds which inhibit activation of a member of the IL-6 signaling pathways are further selected by a biochemical means from the compounds selected by the screening step or the like described above.

In an embodiments of the biochemical screening step of the method of the present invention, the biochemical means is any method selected from the group consisting of Western blotting, kinase assay, transcriptional activity assay, surface plasmon resonance, immunoprecipitation, cell staining, gene expression analysis using gene chips, Northern blotting, and RT-PCR or a combination thereof.

The “biochemical means” as used herein refers to a means that can be used in studies in the biochemical field, including immunological means and physical means. The “immunological means” refers to a means using an antibody, such as Western blotting, immunoprecipitation, cell staining, etc. For example, Western blotting allows determination of the phosphorylation state of a member of the IL-6 signaling pathways or an increase or decrease in the amount of a protein in the nucleus. The “physical means” refers to a means using a physiological property of a protein or compound or the like to determine a biochemically important parameter such as their binding intensity, e.g., surface plasmon resonance.

Surface plasmon resonance is a means making it possible to analyze the dynamics of binding of a protein to a compound or the like. Specifically, the reflected light from the interface between an aqueous solution of a compound and the surface of a biosensor coated with an immobilized protein or the like is sensed to detect binding interaction. The extent of the interaction between the compound and the protein can be determined by measuring a minor decrease (resonance signal) in the concentration of the aqueous solution of the compound, which varies when it interacts with the protein. Specifically, a BIAcore system (BIACORE) is typically used, whereby the interaction between a compound of interest and a protein and the dissociation constant (Kd) or the like can be determined.

However, the “biochemical means” is not limited to these methods, but includes known methods for measuring the activity of intracellular signal molecules such as kinase assay, or transcriptional activity assay.

The kinase assay refers to an assay performed to determine the influence of a substance controlling the function of a specific kinase. Thus, it can determine the extent of the inhibition of a protein having kinase activity by a compound. The kinase assay can be performed by adding a compound of interest, a protein having kinase activity, a substrate for the kinase and ATP into an appropriate buffer and identifying the ATP level remaining after kinase reaction for a predetermined period. ATP can be detected by any quantitative method, e.g., by measuring the luminescence level when luciferin is converted into oxidized luciferin by using ATP under the enzymatic action of luciferase. Alternatively, the influence of a compound of interest on kinase activity can be quantified using a fluorescent dye, luminescent dye or radioactive isotope, e.g., using [γ-³²P] ATP to measure the amount of γ-³²P bound to the substrate by means of a scintillation counter.

The transcriptional activity assay refers to an assay for analyzing the regulation of the transcription level of a gene or an assay for determining the activation of a transcription factor activating a target gene or any member upstream of it. Specifically, known assays include luciferase assay or CAT (chloramphenicol acetyl transferase) assay. These methods involve preparing a plasmid containing the luciferase gene or CAT gene as a reporter downstream of the transcription regulatory region of a target gene and measuring the enzyme activity in a cell transduced with the plasmid in order to determine activation of a member of a signaling pathways leading to transcriptional regulation of the target gene. A specific procedure of luciferase assay is illustrated in the examples.

Immunoprecipitation, cell staining, gene expression analysis using gene chips, Northern blotting, and RT-PCR are routine experimental methods known in the art.

iii) Negative Screening Step

The screening method of the present invention may further comprise, between the positive screening step and the biochemical screening step, a negative screening step using a cell capable of proliferating by IL-6 stimulation to select compounds inhibiting IL-6-dependent proliferation of the cell from the compounds selected in the positive screening step.

In an embodiment of the negative screening step of the present invention, the cell capable of proliferating by IL-6 stimulation used in the negative screening step is preferably derived from humans.

In preferred embodiments of the negative screening step of the present invention, the cell capable of proliferating by IL-6 stimulation used in the negative screening step is an IL-6-dependent tumor cell line.

The “cell capable of proliferating by IL-6 stimulation” refers to a cell capable of proliferating by adding IL-6 at a concentration of, but not limited to, 5 ng/ml, preferably 3 ng/ml, more preferably 1 ng/ml into the culture medium during cell culture.

The cell is preferably derived from humans, more preferably an IL-6-dependent tumor cell line. Still more preferably, it is a human Lennert's T cell lymphoma cell line KT-3 (Non-patent document 12) (obtained from Professor Norihiro Nishimoto, Laboratory of Immune Regulation, Graduate School of Frontier Biosciences, Osaka University, Japan) or a human myeloma cell line INA-6 (Non-patent document 13) (obtained from Martin Gramatzki, M.D., Director, Division of Stem Cell and Immunotherapy 2nd Medical Department, University of Kiel Schittenhelmstr. 1224105 Kiel Germany).

In the negative screening step, IL-6 is added at a concentration of 1-20 ng/ml, preferably 2-10 ng/ml, more preferably 5 ng/ml into a medium in which a cell capable of proliferating by IL-6 stimulation is to be cultured. Then, each compound is added at a concentration of 0.1-50 μM, preferably 1-25 μM, more preferably 2-10 μM. Compounds which inhibit activation of a member of the IL-6 signaling pathways can be further screened by selecting compounds inhibiting the proliferation of the cell from the compounds selected by positive screening.

The expression “inhibiting IL-6-dependent proliferation of a cell” means that the proportion of grown cells as compared with the cell proliferation in a control experiment is at least 50% or less, preferably 25% or less, more preferably 10% or less, still more preferably 1% or less. The cell proliferation rate can be determined by a method using FACS (described in the examples) or measuring the uptake of bromodeoxyuridine (BrdU) or the like.

Conventionally, this negative screening step took place first, whereby compounds inhibiting cell proliferation by the cytotoxicity of the compounds were also selected. However, this possibility is eliminated in the present invention by performing this negative screening step after the positive screening step.

In some preferred embodiments of the negative screening step of the present invention, the cell capable of proliferating by IL-6 stimulation is derived from humans. This is important for determining whether or not the compounds selected by the positive screening step are compounds inhibiting activation of a member of the IL-6 signaling pathways in human cells when the positive screening step is performed in non-human cells.

### II. Compounds Inhibiting Activation of a Member of the IL-6 Signaling Pathways

The present invention provides compounds which are identified by the screening methods described above, and inhibit activation of a member of the IL-6 signaling pathways.

The compounds inhibiting activation of a member of the IL-6 signaling pathways have a structural formula shown in Formulae (1)-(6) below:

### Formula (1):

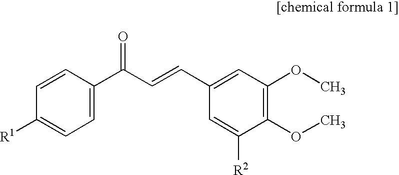

wherein
R¹ represents hydrogen, (C₁-C₆)alkyl or halogen; and
R² represents hydrogen, (C₁-C₆)alkyl, hydroxy or (C₁-C₆)alkoxy;

### Formula (2):

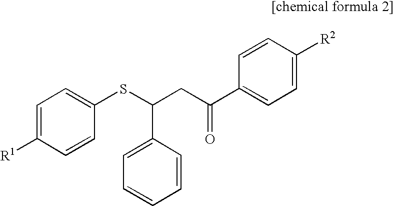

wherein
R¹ represents hydrogen, (C₁-C₆)alkyl, or halogen; and
R² represents hydrogen, (C₁-C₆)alkyl, hydroxy or (C₁-C₆)alkoxy;

### Formula (3):

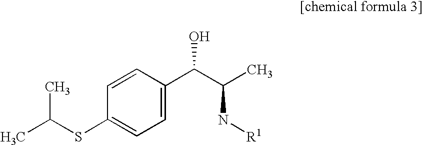

wherein
R¹ represents hydrogen or (C₁-C₁₀)alkyl;

### Formula (4):

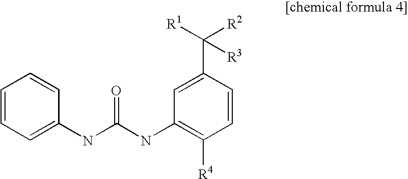

wherein
R¹, R², and R³ are identical or different and each represents hydrogen or halogen; and
R⁴ represents hydrogen or halogen;

### Formula (5):

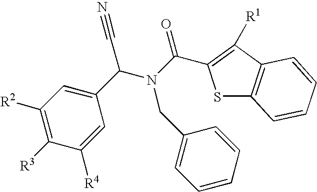

wherein
R¹ represents hydrogen or halogen; and
R², R³ and R⁴ are identical or different and each represents hydrogen, (C₁-C₆)alkyl, hydroxy or (C₁-C₆)alkoxy; and

### Formula (6):

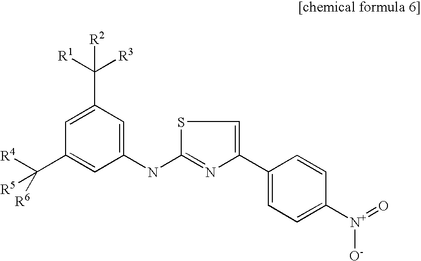

wherein
R¹, R², and R³ are identical or different and each represents hydrogen or halogen; and
R⁴, R⁵, and R⁶ are identical or different and each represents hydrogen or halogen.

In the examples described below, the following compounds were actually selected as compounds which inhibit activation of STAT3 among members of the IL-6 signaling pathways (Compounds Nos. 1, 2, 7, 10, 13, 14 and 16).

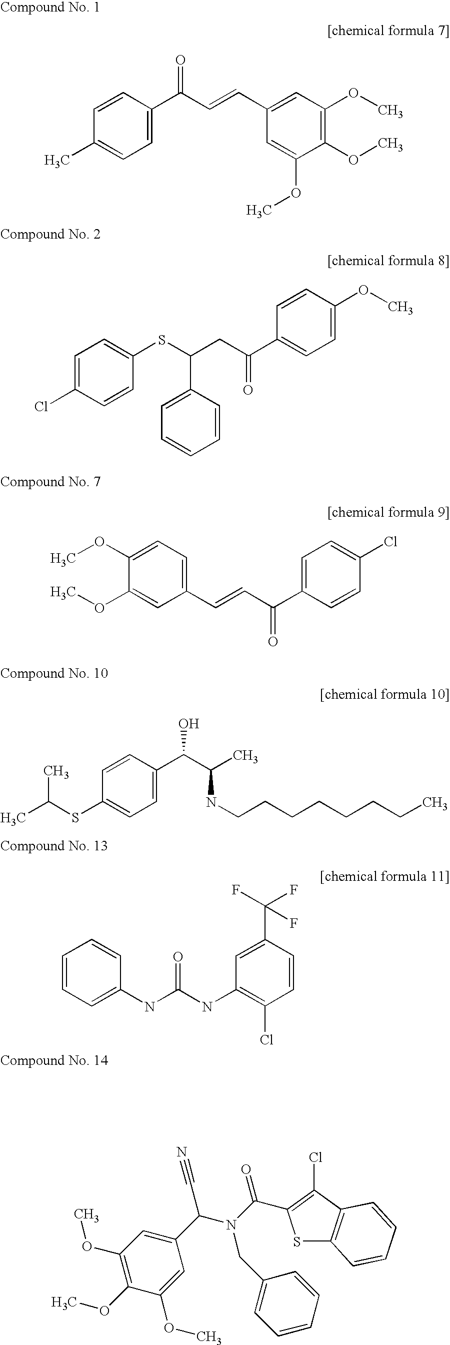

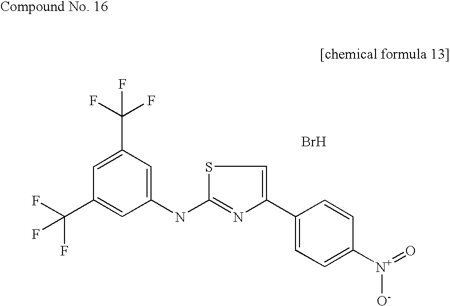

In a preferred compound of Formula (1), therefore, R¹ is methyl, and R² is methoxy. In an alternative preferred compound, R¹ is chlorine, and R² is hydrogen.

In a preferred compound of Formula (2), R¹ is chlorine, and R² is methoxy.

In a preferred compound of Formula (3), R¹ is n-octyl.

In a preferred compound of Formula (4), R¹, R², and are fluorine, and R⁴ is chlorine.

In a preferred compound of Formula (5), R¹ is chlorine, and R², R³, and R⁴ are methoxy.

In a preferred compound of Formula (6), R¹, R², and R³ are fluorine, and R⁴, R⁵, and R⁶ are also fluorine.

### BRIEF DESCRIPTION OF THE DRAWINGS

FIG. 1 shows the IL-6 signaling pathways.

FIG. 2 shows that Compound Nos. 1, 2, 4, 7, 10, 13, 14 and 16 inhibit IL-6-dependent proliferation of KT-3 cells. Specifically, it shows the results of FACS analysis of the inhibitory activity of each compound against the proliferation of KT-3 cells in the negative screening step. Grown cells are plotted in areas enclosed by black lines, and the proportions of the total are shown in % next to the plots. The concentration used were 5 ng/ml for IL-6, 2 μM for Compound No. 4, and 10 μM for other compounds.

FIG. 3 shows that Compounds (Nos. 13 and 16) inhibit IL-6-induced tyrosine phosphorylation of STAT3 in M1 cells. Specifically, it shows the results of Western blot analysis of the inhibition of phosphorylation of STAT3 in M1 cells after treatment with 10 μM of Compound No. 1, 2, 7, 10, 13, 14 or 16 for 30 minutes followed by 10 ng/ml IL-6 for 1 hour in the biochemical screening step. Compounds (Nos. 13 and 16) showed obvious inhibition of phosphorylation of STAT3.

FIG. 4 shows that Compounds (Nos. 2, 13 and 16) inhibit IL-6-induced transcriptional activity of STAT3. In the biochemical screening step, cells were assayed after treatment with 20 μM of Compound No. 2, 13, or 16 for 1 hour followed by 10 ng/ml of IL-6 for 16 hours. The results of Western blot analysis of the inhibition of phosphorylation of STAT3 in 293 T cells 17 hours after the treatment with the compounds are shown. Not only Compounds (Nos. 13 and 16 but also No. 2) showed obvious inhibition of phosphorylation of STAT3.

FIG. 5 shows the results of luciferase assay of the inhibition of the transcriptional activity of STAT3 by Compound No. 2, 13, or 16 in 293 T cells in the biochemical screening step. The experimental system is similar to that of FIG. 4. Compounds (Nos. 2, 13 and 16) showed inhibition of IL-6-induced transcriptional activity of STAT3.

FIG. 6 shows that Compound No. 2 inhibits IL-6-induced tyrosine phosphorylation of STAT3, but not phosphorylation of ERK1/2. Inhibitory effect of Compound No. 2 on STAT3 phosphorylation was evaluated with time in the biochemical screening step. The concentration used were 10 ng/ml for IL-6 and 5 μM for Compound No. 2, and D means DMSO.

FIG. 7 shows that Compounds (Nos. 13 and 16) inhibit IL-6-induced tyrosine phosphorylation of STAT3. Inhibitory effect of Compound Nos. 13 and 16 on STAT3 phosphorylation was evaluated in view of time curve in the biochemical screening step. The concentration used were 10 ng/ml for IL-6 and 10 μM for the compounds, and D means DMSO.

FIG. 8 shows that tyrosine phosphorylation of various proteins and phosphorylation of ERK1/2 were induced in M1 cells after treatment with Compound No. 4. The cells were treated with 2 μM of Compound No. 4 for 14 hours.

FIG. 9 shows that no phosphorylation of MEK, p38MAPK, JNK and Akt was induced in M1 cells by Compound No. 4. The cells were treated with 2 μM of Compound No. 4 for 14 hours.

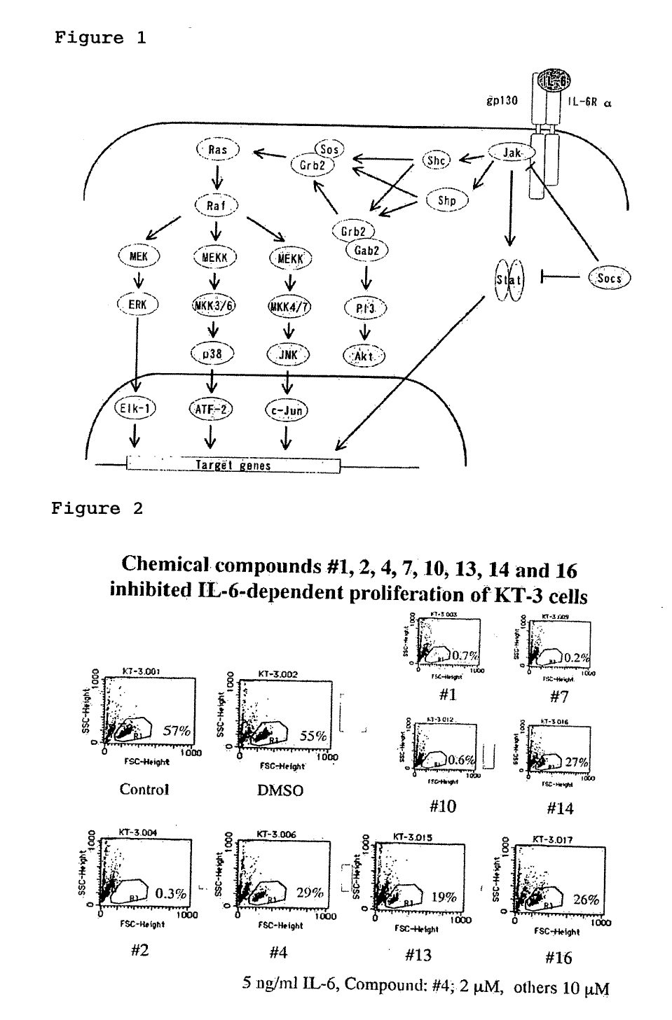

### EXAMPLES

The following examples are provided for illustrative purposes only and should not be construed to limit the present invention. The scope of the present invention is determined on the basis of the claims. Those skilled in the art can readily add modifications/changes in the light of the description herein.

### I. Positive Screening (First Screening)

### Preparation of M1 Clones

Two hundred clones of M1 murine leukemia cells were isolated by limiting dilution in RPMI medium supplemented with 10% fetal calf serum (FCS). Among them, the most rapidly proliferating 20 clones were subsequently isolated. They were evaluated by FACS after incubation with 5 ng/ml murine IL-6 in RPMI medium supplemented with 10% fetal calf serum (FCS) for 72 hours, and cells most susceptible to death (viability 1% or less) were selected.

### Screening Procedure

M1 murine leukemia cells were plated at 2000 cells/well on 384-well plates in 50 μl of RPMI medium containing 10% fetal calf serum (FCS), supplemented with 2 μl of 1 mM DMSO solution alone (Blank) or 2 μl of 1 mM DMSO solution containing each of 12700 compounds (final concentration 2 μM), and murine IL-6 at a final concentration of 10 ng/ml. After incubation for 72 hours, 5 μl of Cell Counting WST-8 kit solution (LIFE SCIENCE REAGENTS SERIES-3) was added. After incubation for 2 hours, the absorbance was measured at 450 nm (OD 450) to calculate the number of viable cells. This primary high-throughput screening yielded positive results in 74 compounds. After incubation with these compounds directly dissolved at 4 stages of final concentrations of 20/5/1.25/0.3 μM without using DMSO and murine IL-6 at a final concentration of 10 ng/ml, the number of viable cells was evaluated in the same manner as in the primary screening. Samples treated with each compound without murine IL-6 were also prepared and measured for the number of viable cells after incubation for 72 hours. By this means, the cytotoxicity of each compound was evaluated. As a result, 18 of the 74 compounds were found to inhibit IL-6-induced apoptosis at non-cytotoxic concentrations and determined to be positive in the first screening.

### II. Negative Screening (Second Screening)

Negative screening was performed on the 18 compounds (Nos. 1-18) that were predicted to inhibit activation of a member of the IL-6 signaling pathways by the first screening.

### Screening Procedure

Human tumor cell line KT-3 cells known to have the property of proliferating by IL-6 stimulation were used for negative screening.

KT-3 cells were grown to confluency, and plated on 6-well plates at 1×10⁵ cells/well in RPMI medium supplemented with 10% fetal calf serum (FCS) containing human IL-6 (5 ng/ml). After incubation for 3 hours, dimethyl sulfoxide (DMSO) and the 18 compounds (Nos. 1-18) each at a final concentration of 10 μM (except for No. 4 at 2 μM) were added, and incubation was continued for 48 hours.

### Analysis by Flow Cytometry

The human tumor cell line KT-3 cells treated as above were suspended at about 1×10⁶ cells/100 μl in RPMI medium supplemented with 10% FCS. Then, FSC/SSC dot plots of these cells were obtained by using FACS Calibur (Becton Dickinson).

FIG. 2 shows an example of the results. Grown cells are shown in areas enclosed by black lines. Cells treated with neither compound nor solvent (control) and cells treated with DMSO alone used as the solvent for the compounds showed cell proliferation of 57% and 55%, respectively. However, Compounds (Nos. 1, 2, 4, 7, 10, 13, 14 and 16) showed inhibition of cell proliferation.

### Results

Among the 18 compounds (Nos. 1-18) selected by the first screening, 8 compounds (Nos. 1, 2, 4, 7, 10, 13, 14 and 16) were selected by this negative screening step.

### III. Biochemical Screening (Third Screening)

### Western Blotting (M1 Cells)

Exponentially proliferating M1 cells cultured in RPMI medium supplemented with 10% fetal calf serum (FCS) in culture dish were treated with dimethyl sulfoxide (DMSO) and Compound No. 1, 2, 7, 10, 13, 14 or 16 at a final concentration of 10 μM for 30 minutes. Then, the cells were divided into two equal parts and incubated with or without murine IL-6 at a final concentration of 10 ng/ml for further 1 hour. Then, the M1 cells were suspended at a density of 1×10⁷ cells/ml in lysis buffer (0.5% Triton X-100, 50 mM Tris-HCl, (pH7.5), 0.1 mM EDTA, 150 mM NaCl, 200 μM Na₃VO₄, 50 mM NaF, 1 mM dithiothreitol (DTT), 0.4 mM phenylmethylsulfonyl fluoride (PMSF), 3 μg/mL aprotinin, 2 μg/mL pepstatin A, 1 μg/mL leupeptin) and placed on ice for 30 minutes. The precipitate was removed by centrifuging the cell lysate at 12000 g for 15 minutes.

The samples thus obtained were separated by sodium dodecyl sulfate-polyacrylamide gel electrophoresis (SDS-PAGE). After the electrophoresis, the gel was placed on a PVDF transfer membrane prewetted in methanol for 10-20 seconds and then soaked in transfer buffer (25 mM tris, 192 mM glycine, 10% methanol, 0.1% SDS), and the assembly was sandwiched between two layers each consisting of 5 sheets of filter paper soaked in transfer buffer, whereby proteins were transferred to the transfer membrane using a semi-dry transfer method.

After transfer, the transfer membrane was shaken in blocking buffer (5% BSA, 1% BSA, 0.1% NaN₃) for 30 minutes or more. Then, the membrane was washed with PBST (137 mM NaCl, 2.7 mM KCl, 1.5 mM KH₂PO₄, 8.1 mM Na₂HPO₄, 0.05% Tween20) three times for 5 minutes by shaking. The membrane was shaken for 60 minutes in an anti-STAT3 polyclonal antibody (C-20, Santa Cruz Biotechnology, Santa Cruz, Calif.) and an anti-phosphorylated STAT3 monoclonal antibody (B-7, Santa Cruz Biotechnology) as primary antibodies both diluted 1:1000 in PBST.

Then, the membrane was washed with PBST three times for 5 minutes by shaking. The membrane was shaken for 30 minutes in a 1:5000 dilution of an HRPO-conjugated anti-rabbit or mouse IgG antibody as a secondary antibody. Then, the membrane was washed with PBST three times for 5 minutes by shaking.

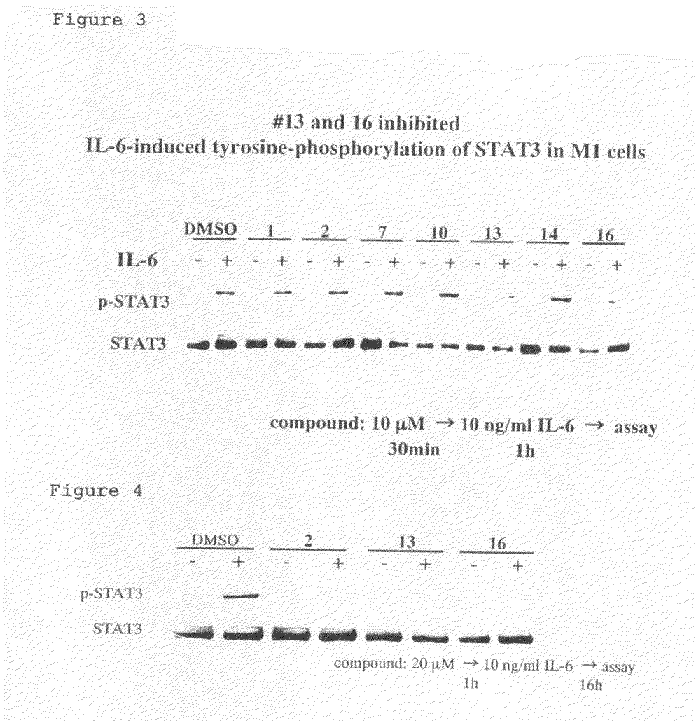

Then, a chromogenic step was performed by a known chromogenic method to detect bands. The results are shown in FIG. 3. Evaluation of tyrosine phosphorylation of STAT3 revealed obvious inhibition of tyrosine phosphorylation of STAT3 by Compounds No. 13 and 16.

### Western Blotting (293 T Cells)

Exponentially proliferating 293T cells cultured in Dulbecco's Modified Eagle's Medium (DMEM) supplemented with 10% fetal calf serum (FCS) in culture dish were incubated with dimethyl sulfoxide (DMSO) and Compounds No. 1, 2, 4, 7, 10, 13, 14 or 16 at a final concentration of 20 μM for 1 hour. Then, the cells were divided into two equal parts and incubated with or without murine IL-6 at a final concentration of 10 ng/ml for further 16 hours. Then, the 293T cells were suspended at a density of 1×10⁷ cells/ml in lysis buffer, and placed on ice for 30 minutes. The precipitate was removed by centrifuging the cell lysate at 12000 g for 15 minutes.

The subsequent Western blot step was performed by the method described above. The results are shown in FIG. 4. Evaluation of tyrosine phosphorylation of STAT3 revealed obvious inhibition of tyrosine phosphorylation of STAT3 by Compounds No. 2, 13 and 16.

### Luciferase Assay

A reporter vector (2 μg) containing the firefly luciferase gene linked downstream of the glial fibrillary acidic protein (GFAP) gene promoter sequence and an internal control vector (0.1 μg) containing the EF1 gene promoter linked to the Renilla luciferase gene were transfected into 293T cells at 1×10⁶ cells/well in 12-well plates using Lipofectamine Plus Reagent (Life Technologies, Bethesda, Md.) as instructed by the manufacturer. Twenty-three hours after the transfection, dimethyl sulfoxide (DMSO) and Compounds No. 1, 2, 4, 7, 10, 13, 14 and 16 each at a final concentration of 20 μM were added. One hour after the compounds were added, the cells treated with each compound were divided into two equal parts and stimulated or not with human IL-6 at a final concentration of 10 ng/ml. After 16 hours, cell extracts were prepared from the cells and measured for the specific activity of firefly luciferase to Renilla luciferase using Dual luciferase reporter system (Promega, Madison, Wis.).

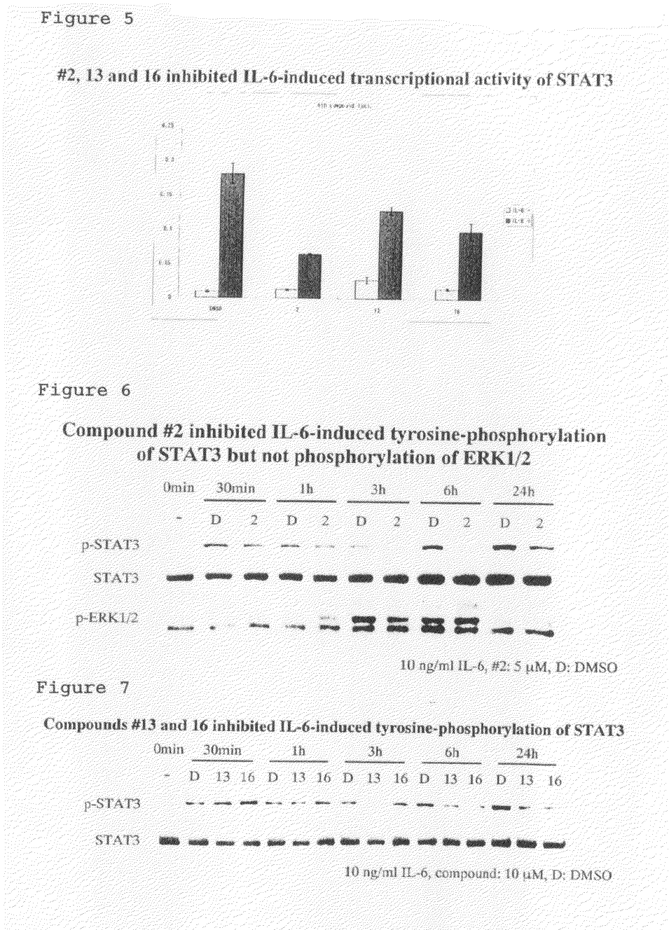

The results are shown in FIG. 5. Compounds No. 2, 13 and 16 showed an obvious inhibitory effect on the transcriptional activity of STAT3.

Analysis of Compound No. 2 DMSO alone or Compound No. 2 at a final concentration of FM was added to exponentially proliferating M1 cells cultured in RPMI medium supplemented with 10% fetal calf serum (FCS) in culture dish. Immediately after, murine IL-6 was added at a final concentration of 10 ng/ml, and cell extracts were prepared at a density of 1×10⁷ cells/ml after 0, 30 minutes, 1, 3, 6, and 24 hours.

Each sample was evaluated for tyrosine phosphorylation of STAT3 according to the Western blot method described above. The results are shown in FIG. 6. The cells treated with Compound No. 2 showed obvious inhibition of tyrosine phosphorylation of STAT3 at 3, 6, and 24 hours after the compound was added. On the other hand, Compound No. 2 was found to have no influence on the phosphorylation of ERK1/2, which is a different pathway from the STAT3-mediated pathway induced by IL-6 stimulation. This result suggests that Compound No. 2 specifically inhibits the STAT3-mediated pathway.

### Analysis of Compounds No. 13, 16

Similar experiments were performed on Compounds No. 13, 16 (final concentration 10 μM) in the same manner as for Compound No. 2. The results are shown in FIG. 7. The cells treated with Compounds No. 13, 16 showed inhibition of tyrosine phosphorylation of STAT3 at 3 hours or later after the compounds were added.

### Analysis of Compound No. 4

Compound No. 4 had a steroid skeleton. Compounds having such a skeleton are known to have the activity of non-specifically inhibiting cell differentiation or death.

The effect of Compound No. 4 on tyrosine phosphorylation of several proteins was evaluated.

Specifically, M1 cells were incubated with Compound No. 4 at a final concentration of 2 μM for 14 hours. Then, tyrosine phosphorylation of the proteins was evaluated by a method similar to the Western blot method described above.

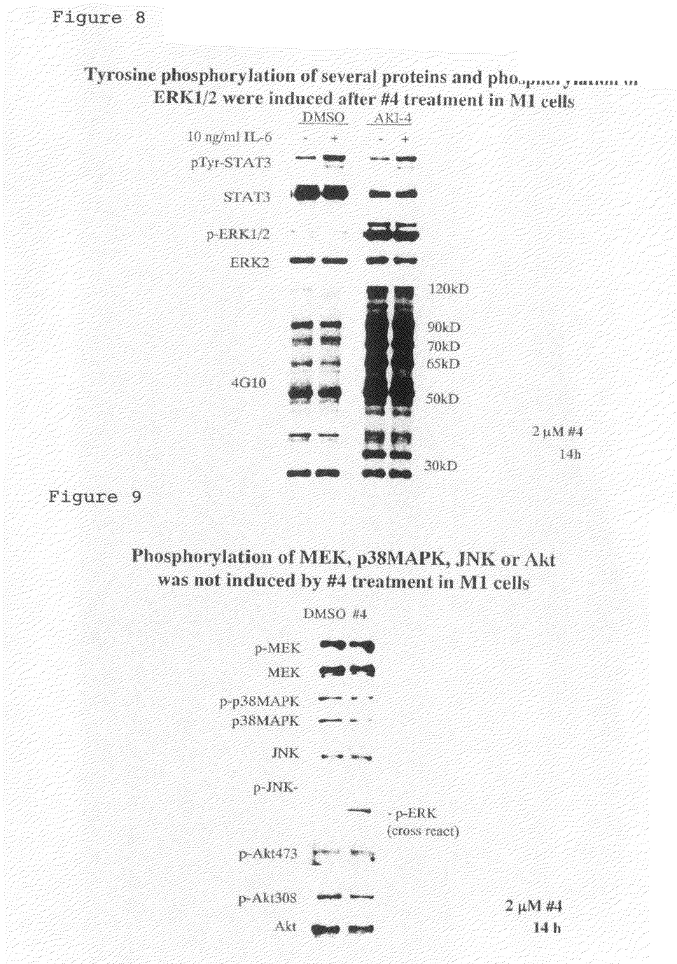

The results are shown in FIG. 8. The presence of Compound No. 4 did not alter the protein level of tyrosine phosphorylated STAT3, but the presence of Compound No. 4 remarkably increased the protein level of tyrosine phosphorylated ERK1/2. When a monoclonal antibody 4G10 specifically binding to phosphorylated tyrosine was used, it was shown that Compound No. 4 non-specifically remarkably promoted tyrosine phosphorylation of proteins.

Tyrosine phosphorylation of different proteins was evaluated in a similar experimental system. The results are shown in FIG. 9. Compound No. 4 did not promote tyrosine phosphorylation of MEK, p38MAPK, JNK, Akt. However, it promoted tyrosine phosphorylation of ERK in the same manner as in the experiments above.

ERK is a kinase known to be involved in cell proliferation and cell viability. Thus, Compound No. 4 inhibited apoptosis of M1 clones by non-specifically activating ERK in the positive screening, indicating that it is not a compound specifically inhibiting the activation of a member of the IL-6 signal transduction system aimed by the present invention, but an example of a rare background in the present screening method.

### Outputs of the Present Screening

Seven compounds, i.e., Compounds No. 1, 2, 7, 10, 13, 14 and 16 were selected using the positive and negative screening steps of the present screening method. The biochemical screening step of the present example was designed to analyze primarily STAT3 in the IL-6 signaling pathways and revealed that at least Compounds Nos. 2, 13 and 16 influence the phosphorylation of STAT3. Other compounds were also selected using the positive and negative screening steps, suggesting that they inhibit activation of a member of the IL-6 signaling pathways, including the STAT3-mediated pathway, Ras-mediated pathway, and PI3K- and Akt-mediated pathway.

### Discussion

According to the present invention, methods for screening compounds which inhibit activation of a member of the IL-6 signaling pathways were provided, and compounds which inhibit activation of a member of the IL-6 signaling pathways were actually obtained. Additional compounds which inhibit activation of a member of the IL-6 signal pathways can be identified by screening other compound libraries using the methods of the present invention. Moreover, the compounds thus identified are expected to provide drugs for diseases in which the IL-6 signaling pathways are involved, such as cancer.

1. A method for screening compounds which inhibit activation of a member of the IL-6 signaling pathways, comprising:
(a) a positive screening step using a cell capable of being killed by IL-6 stimulation to select compounds which inhibit death of the cell when stimulated by IL-6; and then
(b) a biochemical screening step to further select compounds inhibiting activation of a member of the IL-6 signaling pathways by a biochemical means from the compounds selected in step (a).

2. The method of claim 1 wherein the cell capable of being killed by IL-6 stimulation used in the positive screening step (a) is derived from mice or humans.

3. The method of claim 1 or 2 wherein the cell capable of being killed by IL-6 stimulation used in the positive screening step (a) is a clone derived from murine M1 cells.

4. The method of claim 1 or 2 wherein the cell capable of being killed by IL-6 stimulation used in the positive screening step (a) is a genetically engineered cell.

5. The method of claim 1 wherein a target molecule of the compounds which inhibit activation of a member of the IL-6 signaling pathways is a member of the STAT3-mediated pathway.

6. The method of claim 1 wherein a target molecule of the compounds which inhibit activation of a member of the IL-6 signaling pathways is a member of the RAS-mediated pathway.

7. The method of claim 1 wherein a target molecule of the compounds which inhibit activation of a member of the IL-6 signaling pathways is a member of the PI3K- and Akt-mediated pathway.

8. The method of claim 1 wherein the biochemical means in the biochemical screening step (b) is any method selected from the group consisting of Western blotting, kinase assay, transcriptional activity assay, surface plasmon resonance, immunoprecipitation, cell staining, gene expression analysis using gene chips, Northern blotting, and RT-PCR or a combination thereof.

9. The method of claim 1, further comprising, between the positive screening step (a) and the biochemical screening step (b), a negative screening step using a cell capable of proliferating with IL-6 stimulation to select compounds inhibiting IL-6-dependent proliferation of the cell from the compounds selected in step (a).

10. The method of claim 8 wherein the cell capable of proliferating with IL-6 stimulation used in the negative screening step is derived from humans.

11. The method of claim 8 wherein the cell capable of proliferating by IL-6 stimulation used in the negative screening step is an IL-6-dependent tumor cell line.

12. A compound which inhibit activation of a member of the IL-6 signaling pathways, selected from the group consisting of Formulae (1)-(6) below:
Formula (1): 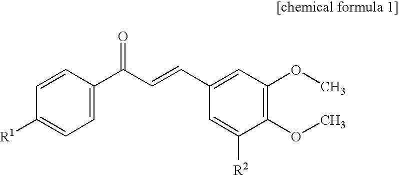
wherein
R¹ represents hydrogen, (C₁-C₆)alkyl, or halogen; and
R² represents hydrogen, (C₁-C₆)alkyl, hydroxy or (C₁-C₆)alkoxy;
Formula (2): 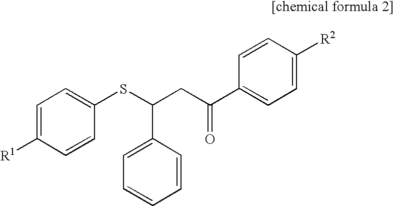
wherein
R¹ represents hydrogen, (C₁-C₆)alkyl, or halogen; and
R² represents hydrogen, (C₁-C₆)alkyl, hydroxy or (C₁-C₆)alkoxy;
Formula (3): 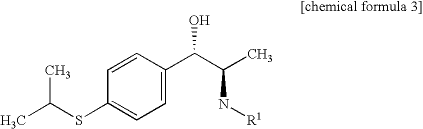
wherein
R¹ represents hydrogen or (C₁-C₁₀)alkyl;
Formula (4): 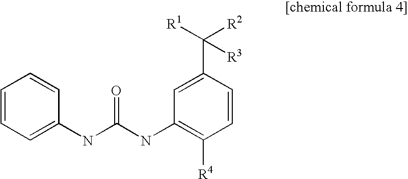
wherein
R¹, R², and R³ are identical or different and each represents hydrogen or halogen; and
R⁴ represents hydrogen or halogen;
Formula (5): 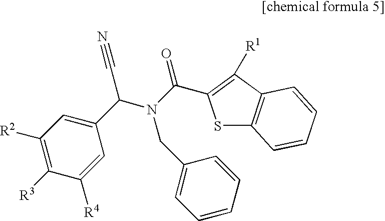
wherein
R¹ represents hydrogen or halogen; and
R², R³ and R⁴ are identical or different and each represents hydrogen, (C₁-C₆)alkyl, hydroxy or (C₁-C₆)alkoxy; and
Formula (6): 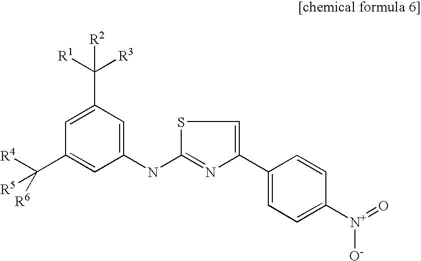
wherein
R¹, R², and R³ are identical or different and each represents hydrogen or halogen; and
R⁴, R⁵, and R⁶ are identical or different and each represents hydrogen or halogen.

13. A compound which inhibit activation of a member of the IL-6 signaling pathways, selected from the group consisting of Formulae (7)-(13) below: 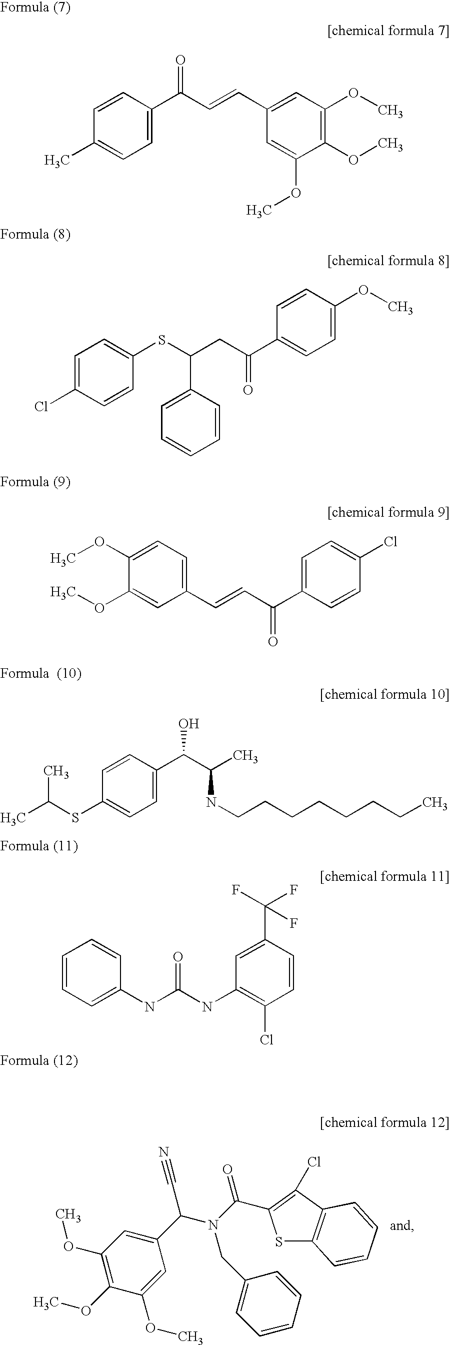 [chemical structure]
